(19) United States
(12) Patent Application Publication
(10) Pub. No.: US 2025/0073049 A1
(43) Pub. Date: Mar. 6, 2025

(54) UNI-COMPARTMENTAL ORTHOPAEDIC SYSTEM HAVING MODELED SURFACES

(71) Applicant: DePuy Ireland Unlimited Company, Ringaskiddy (IE)

(72) Inventors: Mark Reason, Leeds (GB); Mathew Walkington, Leeds (GB); James Brewerton, Leeds (GB); David R. Wolfson, Batley (GB); Alice De Courcy-Grylls, Leeds (GB); Robert Freeman, Leeds (GB)

(21) Appl. No.: 18/813,173

(22) Filed: Aug. 23, 2024

Related U.S. Application Data
(60) Provisional application No. 63/535,127, filed on Aug. 29, 2023.

(51) Int. Cl. A61F 2/50

(52) U.S. Cl. CPC A61F 2/5046; A61F 2240/001

(57) ABSTRACT

A method for designing a uni-compartmental orthopaedic prosthesis includes generating a three-dimensional statistical model of a knee joint, determining a curvature of a section of a bone of the knee joint represented by the three-dimensional statistical model, and designing a section of the uni-compartmental orthopaedic prosthesis corresponding to the section of the bone of the knee joint to have a curvature that matches the curvature of the section of the bone of the knee joint. Additionally, a tibial and femoral uni-compartmental orthopaedic prosthesis are also disclosed, each of which includes a section having a curvature that matches a corresponding section of a corresponding bone of the knee joint represented by the three-dimensional statistical model.

The present application claims priority under 35 U.S.C. § 119 to, and the benefit of, U.S. Provisional Patent Application Ser. No. 63/535,127, entitled “UNI-COMPARTMENTAL ORTHOPAEDIC SYSTEM HAVING MODELED SURFACES,” which was filed on Aug. 29, 2023, the entirety of which is expressly incorporated herein by reference.

### TECHNICAL FIELD

The present disclosure relates to orthopaedic knee prosthesis systems and, more specifically, to uni-compartmental orthopaedic prostheses and technologies for developing such prostheses.

### BACKGROUND

Joint arthroplasty is a well-known surgical procedure by which a diseased and/or damaged natural joint is replaced by a prosthetic joint. A typical knee prosthesis includes a tibial tray, a femoral component, and a polymer insert or bearing positioned between the tibial tray and the femoral component. Additionally, in some cases, the knee prosthesis may also include a patella component. Depending on the severity of the damage to the patient's joint, orthopaedic prostheses of varying mobility may be used. For example, the knee prosthesis may include a “fixed” tibial insert in cases wherein it is desirable to limit the movement of the knee prosthesis, such as when significant soft tissue damage or loss is present. Alternatively, the knee prosthesis may include a “mobile” tibial insert in cases wherein a greater degree of freedom of movement is desired.

Additionally, depending on the condition of the patient's knee joint, the selected orthopaedic knee prosthesis may be embodied as a total knee prosthesis designed to replace the femoral-tibial interface of both condyles of the patient's femur or a uni-compartmental (or uni-condylar) knee prosthesis designed to replace the femoral-tibial interface of a single condyle of the patient's femur. Total knee replacement or arthroplasty may involve replacement of the mid-shaft portion of the femur, proximal, distal, and/or total femur, and proximal tibia. Uni-compartmental knee replacement or arthroplasty involves uni-condylar resurfacing. Uni-compartmental knee arthroplasty provides an alternative to total knee arthroplasty for rehabilitating knees when only one condyle has been damaged as a result of trauma or disease such as non-inflammatory degenerate joint disease or its composite diagnosis of osteoarthritis or post-traumatic arthritis. In some cases, the orthopaedic knee prosthesis may be a bi-compartmental knee prosthesis formed by two uni-compartmental knee prostheses, which replaces each of the medial and lateral femoral condyles and tibial articular surfaces of the patient. The one or pair of uni-compartmental knee prostheses may be configured to articulate with the patient's natural patella or, alternatively, with a prosthetic patella component designed to replace the patient's natural patella.

### SUMMARY

According to one aspect of the present disclosure, a method for designing a uni-compartmental orthopaedic prosthesis may include generating a three-dimensional statistical shape model of a knee joint, determining a curvature of a section of a bone of the knee joint represented by the three-dimensional statistical shape model, and designing the uni-compartmental orthopaedic prosthesis for the bone of the knee joint based on the three-dimensional statistical shape model. The three-dimensional statistical shape model may be indicative of an average knee joint of a pool of patient participants. The uni-compartmental orthopaedic prosthesis may include a section corresponding to the section of the bone of the knee joint represented by the three-dimensional statistical shape model. Additionally, designing the uni-compartmental orthopaedic prosthesis may include defining a curvature of the section of the uni-compartmental orthopaedic prosthesis to match the curvature of the corresponding section of the bone of the knee joint represented by the three-dimensional statistical shape model.

In some embodiments, determining the curvature of the section of the bone of the knee joint may include performing a virtual bone resection on the bone of the knee joint represented by the three-dimensional statistical shape model to determine the curvature. For example, the uni-compartmental orthopaedic prosthesis may be embodied as or otherwise include a tibial uni-compartmental orthopaedic prosthesis. In such embodiments, performing the virtual bone resection may include performing a virtual bone resection in a transverse plane of a proximal end of a tibia of the knee joint represented by the three-dimensional statistical shape model to form a resected section of the tibia in the transverse plane. The curvature of the section may include a lateral external curvature or a medial external curvature of the resected section of the proximal end of the tibia in the transverse plane. In some embodiments, defining the curvature of the section of the uni-compartmental orthopaedic prosthesis may include matching a curvature of an external sidewall of the tibial uni-compartmental orthopaedic prosthesis in the transverse plane to the lateral external curvature or the medial external curvature of the resected section of the tibia in the transverse plane.

Additionally, in some embodiments, the curvature of the section further may include an anterior curvature and a posterior curvature of the resected section of the tibia in the transverse plane. In such embodiments, defining the curvature of the section of the uni-compartmental orthopaedic prosthesis may include matching a curvature of an anterior sidewall of the tibial uni-compartmental orthopaedic prosthesis in the transverse plane to the anterior curvature of the resected section of the tibia in the transverse plane. Additionally or alternatively, defining the curvature of the section of the uni-compartmental orthopaedic prosthesis may include matching a curvature of a posterior sidewall of the tibial uni-compartmental orthopaedic prosthesis in the transverse plane to the posterior curvature of the resected section of the tibia in the transverse plane.

In some embodiments in which the uni-compartmental orthopaedic prosthesis is embodied as or otherwise includes a tibial uni-compartmental orthopaedic prosthesis, determining the curvature of the section of the bone of the knee joint may include determining a curvature of an articular surface of a tibia of the knee joint represented by the three-dimensional statistical shape model in a sagittal plane. Additionally, in such embodiments, defining the curvature of the section of the uni-compartmental orthopaedic prosthesis may include matching a curvature of an articular surface of the tibial uni-compartmental orthopaedic prosthesis in the sagittal plane to the curvature of the articular surface of the tibia of the knee joint represented by the three-dimensional statistical shape model in the sagittal plane.

Additionally, in some embodiments, the uni-compartmental orthopaedic prosthesis may include or otherwise be embodied as a femoral uni-compartmental orthopaedic prosthesis. In such embodiments, determining the curvature of the section of the bone of the knee joint may include performing a virtual bone resection in a transverse plane of a distal end of a femur of the knee joint represented by the three-dimensional statistical shape model to form a resected section of the femur in the transverse plane. The curvature of the section may include an external anterior curvature and an internal anterior curvature of the resected section of the distal end of the femur in the transverse plane. Additionally, in such embodiments, defining the curvature of the section of the uni-compartmental orthopaedic prosthesis may include matching a curvature of an external anterior sidewall of the femoral uni-compartmental orthopaedic prosthesis in the transverse plane to the external anterior curvature of the resected section of the femur in the transverse plane. Additionally or alternatively, defining the curvature of the section of the uni-compartmental orthopaedic prosthesis may include matching a curvature of an internal anterior sidewall of the femoral uni-compartmental orthopaedic prosthesis in the transverse plane to the internal anterior curvature of the resected section of the femur in the transverse plane.

Further, in some embodiments, the uni-compartmental orthopaedic prosthesis may be embodied as or otherwise include a femoral uni-compartmental orthopaedic prosthesis. In such embodiments, determining the curvature of the section of the bone of the knee joint represented by the three-dimensional statistical shape model may include defining a femoral curve formed from a set of distal-most points of a condyle of a femur of the knee joint represented by the three-dimensional statistical shape model. Each of the distal-most points defines a distal-most point of the condyle of the femur at a corresponding degree of flexion.

In some embodiments, the femoral curve may include a distal section corresponding to a distal section of the condyle of the femur of the knee joint represented by the three-dimensional statistical shape model that is defined by a continually decreasing radius of curvature. Additionally, in such embodiments, defining the curvature of the section of the uni-compartmental orthopaedic prosthesis may include matching a distal curvature, when viewed in a sagittal plane through the range of flexion, of a femoral articular surface of the femoral uni-compartmental orthopaedic prosthesis to the distal section of the femoral curve of the femur of the knee joint represented by the three-dimensional statistical shape model.

Additionally or alternatively, the femoral curve may include a posterior section corresponding to a posterior section of the condyle of the femur of the knee joint represented by the three-dimensional statistical shape model that is defined by a two-dimensional radius. The femoral curve may also include a mid-flexion section, located between the distal section and the posterior section, corresponding to a mid-flexion section of the condyle of the femur of the knee joint represented by the three-dimensional statistical shape model that is defined by a two-dimensional spline curve. In such embodiments, defining the curvature of the section of the uni-compartmental orthopaedic prosthesis may include matching a curvature of a posterior section of a femoral articular surface of the femoral uni-compartmental orthopaedic prosthesis to the posterior section of the femoral curve of the femur of the knee joint represented by the three-dimensional statistical shape model. Additionally or alternatively, defining the curvature of the section of the uni-compartmental orthopaedic prosthesis may include matching a curvature of a mid-flexion section of the femoral articular surface of the femoral uni-compartmental orthopaedic prosthesis, located between the distal section and the posterior section, to the mid-flexion section of the femoral curve of the femur of the knee joint represented by the three-dimensional statistical shape model.

According to another aspect of the present disclosure, a tibial uni-compartmental orthopaedic prosthesis may include an anterior end, a posterior end opposite the anterior end, an articular surface extending from the anterior end of the posterior end and configured to articulate with a corresponding condyle of a natural or prosthetic femur, and a bottom surface, opposite the articular surface, extending form the anterior end to the posterior end. The tibial uni-compartmental orthopaedic prosthesis may further include an external sidewall extending from the bottom surface to the articular surface and from the anterior end to the posterior end. The external sidewall may have a curvature, when viewed in a transverse plane, that matches a lateral external curvature or a medial external curvature of a resected section, in the transverse plane, of a proximal end of a tibia represented in a three-dimensional statistical shape model of a knee joint. The three-dimensional statistical shape model may be indicative of an average knee joint of a pool of patient participants.

In some embodiments, the anterior end may include an anterior end sidewall extending from the bottom surface to the articular surface that has a curvature, when viewed in the transverse plane, that matches an anterior curvature of the resected section, in the transverse plane, of the proximal end of the tibia represented in the three-dimensional statistical shape model. Additionally or alternatively, the posterior end may include a posterior end sidewall extending from the bottom surface to the articular surface that has a curvature, when viewed in the transverse plane, that matches a posterior curvature of the resected section, in the transverse plane, of the proximal end of the tibia represented in the three-dimensional statistical shape model.

According to a further aspect of the present disclosure, a femoral uni-compartmental orthopaedic prosthesis may include a bottom surface configured to be coupled to a surgically-prepared distal end of a patient′ femur and a uni-condyle surface opposite the bottom surface. The uni-condyle surface may include a femoral articular surface configured to articulate with a corresponding articular surface a natural or prosthetic tibia. The femoral articular surface may include a femoral curve defined by a first set of distal-most points, each of which defines a distal-most point of the femoral articular surface at a corresponding degree of flexion. The femoral curve may match a virtual femoral curve of a condyle of a femur represented in a three-dimensional statistical shape model of a knee joint. The virtual femoral curve may be defined by a second set of distal-most points, each of which defines a distal-most point of a femoral condyle of the femur at a corresponding degree of flexion. The three-dimensional statistical shape model may be indicative of an average knee joint of a pool of patient participants.

In some embodiments, the femoral uni-compartmental orthopaedic prosthesis may further include an anterior end having an external sidewall and an internal sidewall. Each of the sidewalls may extend from the bottom surface to the uni-condyle surface. The external sidewall may have a curvature, when viewed in a transverse plane, that matches an external anterior curvature of a resected section, in the transverse plane, of a distal end of the femur represented in the three-dimensional statistical shape model of the knee joint. Additionally, the internal sidewall may have a curvature, when viewed in a transverse plane, that matches an internal anterior curvature of a resected section, in the transverse plane, of the distal end of the femur represented in a three-dimensional statistical shape model of a knee joint.

### BRIEF DESCRIPTION OF THE DRAWINGS

The detailed description particularly refers to the following figures, in which:

FIG. 1 is a perspective view of a patient's knee joint having a uni-compartmental knee prosthesis implanted therein;

FIG. 2 is a side elevation view of the uni-compartmental knee prosthesis of FIG. 1;

FIGS. 3A-3C are a flow diagram of a method for designing and manufacturing the uni-compartmental knee prosthesis of FIG. 2;

FIG. 4. is an anterior elevation view of a three-dimensional statistical shape model of a generic knee joint formed from medical images of healthy knee joints of individual patients of a patient group and showing several virtual bone resection lines;

FIG. 5 is an anterior perspective view of a tibia of the three-dimensional statistical shape model of FIG. 4 during a virtual resectioning process;

FIG. 6 is a superior plan view of a uni-compartmental tibial insert of the uni-compartmental knee prosthesis of FIG. 2 including anterior, outboard, and posterior curvatures designed based on the three-dimensional statistical shape model of FIGS. 4 and 5;

FIG. 7 is a sagittal cross-sectional view of the uni-compartmental tibial insert of FIG. 6 taken generally along the line 7-7 of FIG. 6;

FIG. 8 is a side elevation view of a femur of the three-dimensional statistical shape model of FIG. 4 including indicia of the sagittal curvature of a condyle of the femur;

FIG. 9 is a distal end view of the femur of the three-dimensional statistical shape model of FIG. 4 including indicia of the distal curvature of a condyle of the femur;

FIG. 10 is a superior plan view of a uni-compartmental femoral component of the uni-compartmental knee prosthesis of FIG. 2 including internal and external curvatures designed based on the three-dimensional statistical shape model of FIG. 4;

FIG. 11 is a sagittal cross-sectional view of the uni-compartmental femoral component of FIG. 10 taken generally along the line 11-11 of FIG. 10 including a sagittal curvature designed based on the three-dimensional statistical shape model of FIG. 4;

FIG. 12 is an anterior perspective view of the uni-compartmental femoral component of FIG. 10 showing a trochlea groove of the anterior portion of the uni-compartmental femoral component;

FIG. 13 is a coronal cross-sectional view of the uni-compartmental femoral component of FIG. 10 taken generally along the line 13-13 of FIG. 11 including a coronal curvature designed based on the three-dimensional statistical shape model of FIG. 4; and

FIG. 14 is a view of a virtual uni-compartmental tibial insert designed according to the method of FIGS. 3A-3C being virtually implanted into the tibia of the three-dimensional statistical shape model of FIG. 5.

### DETAILED DESCRIPTION OF THE DRAWINGS

While the concepts of the present disclosure are susceptible to various modifications and alternative forms, specific illustrative embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

Terms representing anatomical references, such as anterior, posterior, medial, lateral, superior, inferior, etcetera, may be used throughout the specification in reference to the orthopaedic implants and/or surgical instruments described herein as well as in reference to the patient's natural anatomy. Such terms have well-understood meanings in both the study of anatomy and the field of orthopaedics. Use of such anatomical reference terms in the written description and claims is intended to be consistent with their well-understood meanings unless noted otherwise. Additionally, the term “about” may be used in the specification in reference to certain measurements that are defined within manufacturing tolerances. That is, the provided measurements and/or numerical values may deviate, in practice, due to tolerances inherent in the machine or fabrication process.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

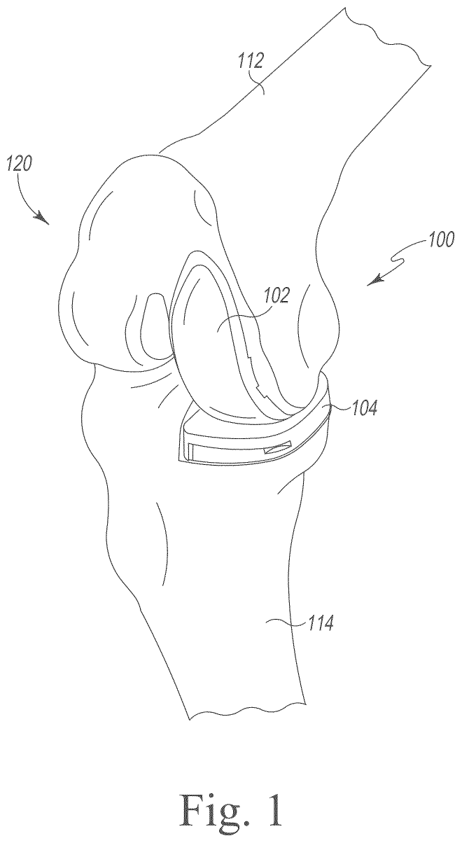

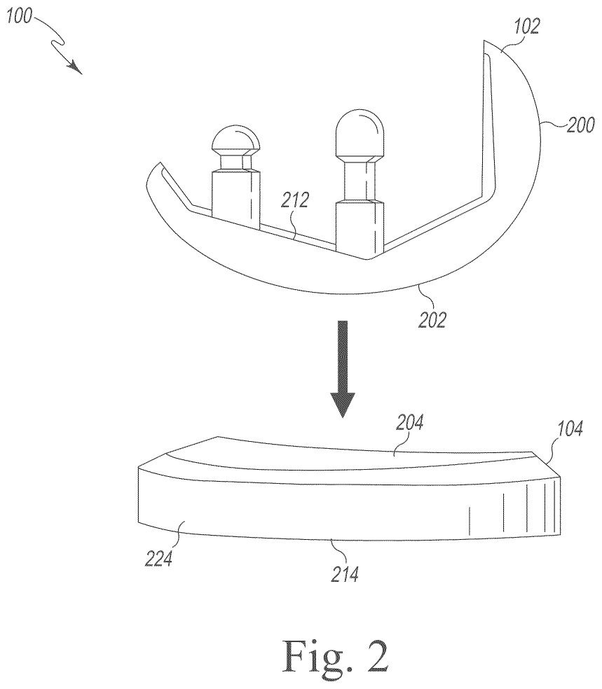

Referring now to FIGS. 1 and 2, in an illustrative embodiment, a uni-compartmental orthopaedic prosthesis 100 includes a femoral uni-compartmental orthopaedic prosthesis 102 and a tibial uni-compartmental orthopaedic prosthesis 104. Additionally, in some embodiments, the uni-compartmental orthopaedic prosthesis 100 may include a uni-compartmental tibial tray (not shown) to which the tibial prosthesis 104 is coupled during use. The femoral uni-compartmental orthopaedic prosthesis 102 (and the uni-compartmental tibial tray if included) are illustratively formed from a metallic material such as cobalt-chromium or titanium, but may be formed from other materials, such as a ceramic material, a polymer material, a bio-engineered material, or the like, in other embodiments. The tibial uni-compartmental orthopaedic prosthesis 104 is illustratively formed from a polymer material such as an ultra-high molecular weight polyethylene (UHMWPE), but may be formed from other materials, such as a ceramic material, a metallic material, a bio-engineered material, or the like, in other embodiments.

As shown in FIG. 1, the femoral uni-compartmental orthopaedic prosthesis 102 is configured to be coupled to a surgically-prepared surface of the distal end of a patient's femur 112 of a patient's knee joint 120. Similarly, the tibial uni-compartmental orthopaedic prosthesis 104 is configured to be coupled to a surgically-prepared surface of the proximal end of the patient's tibia 114, via, for example, a uni-compartmental tibial tray (not shown). Alternatively, in other embodiments, the tibial uni-compartmental orthopaedic prosthesis 104 may be configured to attach to the surgically-prepared surface of the proximal end of the patient's tibia 114 directly, without use of a tibial tray. For example, the tibial uni-compartmental orthopaedic prosthesis 104 and a polymer “tray” may be combined into a single polymeric uni-compartmental component.

In use, as shown in FIG. 1 and indicated in FIG. 2, the femoral uni-compartmental orthopaedic prosthesis 102 is configured to articulate with the tibial uni-compartmental orthopaedic prosthesis 104. To do so, the femoral uni-compartmental orthopaedic prosthesis 102 includes a uni-condyle surface 200, which has a femoral articular surface 202. The femoral uni-compartmental orthopaedic prosthesis 102 also includes a bottom surface 212, opposite the uni-condyle surface 200, which is configured to be secured to the surgically-prepared surface of the distal end of the patient's femur 112 (e.g., via use of bone adhesive or other attachment means) as shown in FIG. 1.

Similarly, the tibial uni-compartmental orthopaedic prosthesis 104 includes a tibial articular surface 204 on which the femoral articular surface 202 (or the patient's natural femoral condyle if no femoral uni-compartmental orthopaedic prosthesis is used) is configured to articulate during normal patient use. The tibial uni-compartmental orthopaedic prosthesis 104 also includes a bottom surface 214, which is configured to couple with a corresponding uni-compartmental tibial tray if used or to the surgically-prepared surface of the patient's tibia 114 if no tibial tray is used. A sidewall 224 extends from the bottom surface 214 to the tibial articular surface 204 and generally defines a thickness of the tibial uni-compartmental orthopaedic prosthesis 104.

As described in more detail below, each of the femoral uni-compartmental orthopaedic prosthesis 102 and the tibial uni-compartmental orthopaedic prosthesis 104 is designed to accommodate a wide audience of patient's. To do so, each of the femoral uni-compartmental orthopaedic prosthesis 102 and the tibial uni-compartmental orthopaedic prosthesis 104 include specific curvatures that match or are otherwise based on corresponding curvatures of an “average” femur and/or tibia. That is, the relevant curvatures of the femoral uni-compartmental orthopaedic prosthesis 102 and the tibial uni-compartmental orthopaedic prosthesis 104 are matched or based on corresponding curvatures of a virtual femur and tibia, respectively, represented in a statistical shape model. The statistical shape model, which may be embodied as a three-dimensional statistical shape model, approximates the average boney anatomy of a pool of patients. The statistical shape model may be developed or generated using one of a number of different methodologies. For example, in the illustrative embodiment, the statistical shape model is developed based on a collection of medical images of “healthy” knee joints taken from a pool of healthy patients and parametrized to allow “morphing” of the shape model by adjusting the associated parameters such that resulting three-dimensional model approximates (e.g., size and shape) healthy knee joints of varying sizes. In this way, each of the femoral uni-compartmental orthopaedic prosthesis 102 and the tibial uni-compartmental orthopaedic prosthesis 104 may be designed to include curvatures, based on corresponding curvatures of the generated three-dimensional statistical shape model, that replicate the natural boney anatomy of average patients across a range of sizes. As such, the produced uni-compartmental orthopaedic prosthesis 100 may provide a better anatomical “fit” to a particular patient because the prosthesis 100 was designed based on an average patient having similar bone sizes.

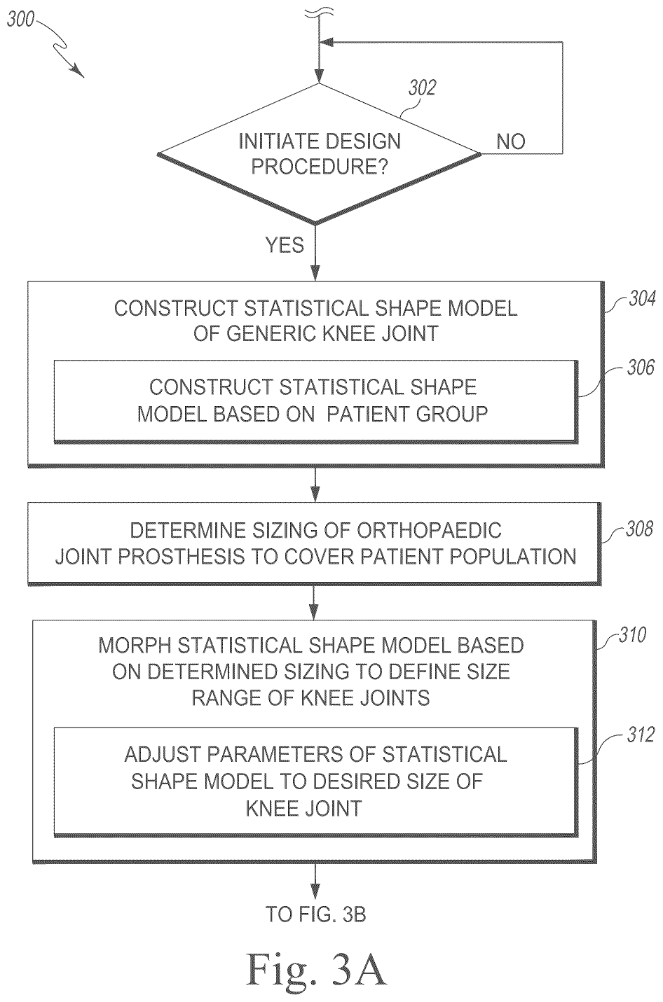

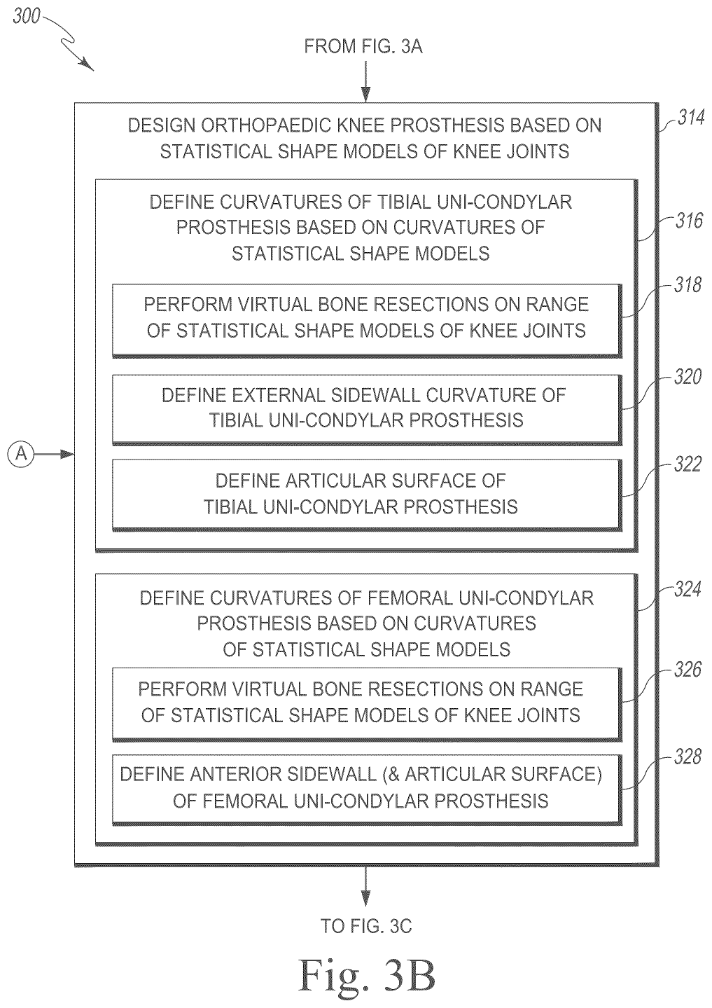

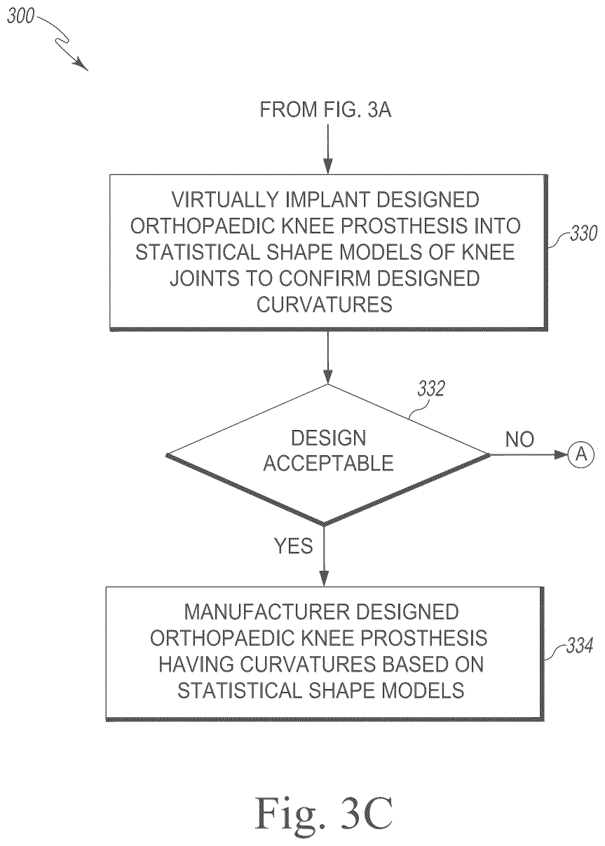

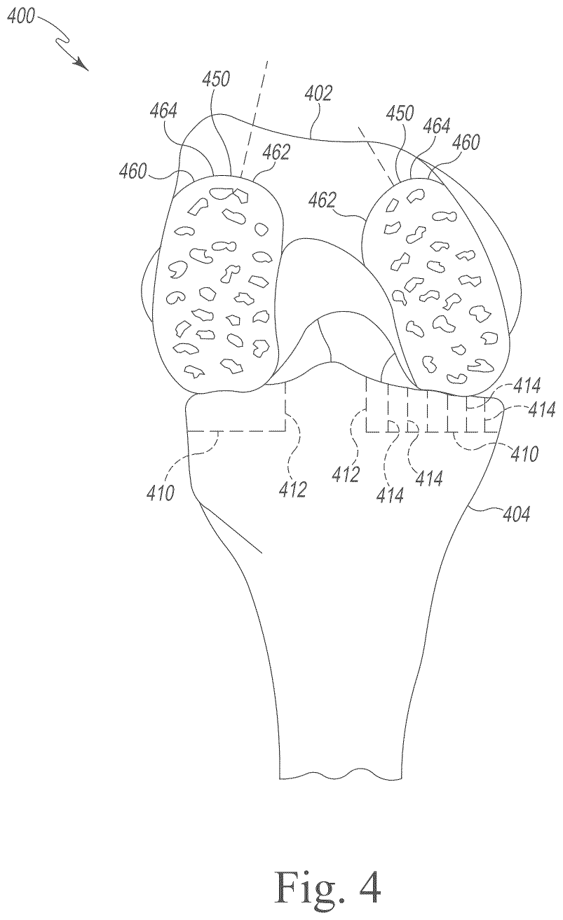

Referring now to FIGS. 3A-3C, each of the femoral uni-compartmental orthopaedic prosthesis 102 and the tibial uni-compartmental orthopaedic prosthesis 104 of the uni-compartmental orthopaedic prosthesis 100 may be designed using a method 300. The method 300 begins with block 302 in which it is determined whether to initiate the design of the uni-compartmental orthopaedic prosthesis 100. If so, the method 300 advances to block 304 in which a statistical shape model (e.g., a three-dimensional statistical shape model) of an average or generic knee joint is constructed. As discussed above, a statistical shape model is a parametrized mathematical model that approximates an average knee joint. As such, in block 306, the statistical shape model is constructed based on a group or pool of patient participants. The statistical shape model may be generated using any suitable methodology. For example, in the illustrative embodiment as shown in FIG. 4, a three-dimensional statistical shape model 400 is generated based on medical images of the relevant knee joint of each patient of the patient group. In this way, the resulting three-dimensional statistical shape model 400 provides a model of an average or generic knee joint, which may be sized based on the associated parameters to represent boney anatomy of people of varying sizes.

Referring back to FIG. 3A, in block 308, a desired range of sizes of the uni-compartmental orthopaedic prosthesis 100 is determined. For example, a size range of 1 to 10 may be used. The sizing is selected so as to cover a large population of potential patients while quantizing the available sizes to reduce the inventory footprint. Of course, any granularity of sizes may be used in various embodiments, depending on the desired inventory footprint and adaptability of the resulting uni-compartmental orthopaedic prosthesis 100 to patent anatomical size variations within each quantized size bucket.

After the range of sizes has been determined in block 308, the three-dimensional statistical shape model 400 is morphed to the desired size for each size within the range of sizes determined in block 308. To do so, in block 312, various parameters of the three-dimensional statistical shape model 400 may be adjusted to morph the three-dimensional statistical shape model 400 to the desired size for each of the determined sizes. In this way, the three-dimensional statistical shape model 400 can be adapted to represent an average boney anatomy of a patient of a corresponding size.

After the range of sizes has been determined in block 308 and the three-dimensional statistical shape model 400 has been morphed to the appropriate size for each size within the determined range in block 310, the method 300 advances to block 314 of FIG. 3B. In block 314, the uni-compartmental orthopaedic prosthesis 100 is designed based on the three-dimensional statistical shape model 400. For example, in block 316, particular curvatures of each size of the tibial uni-compartmental orthopaedic prosthesis 104 may be defined based on corresponding curvatures of the three-dimensional statistical shape model 400. To do so, in the illustrative embodiment, virtual bone resections are performed on the three-dimensional statistical shape model 400 in block 318, which help define or indicate the shape of the curvature of interest. The virtual bone resections are representative of the actual bone cuts performed by an orthopedic surgeon to prepare a patient's tibia for receiving a tibial uni-compartmental orthopaedic prosthesis.

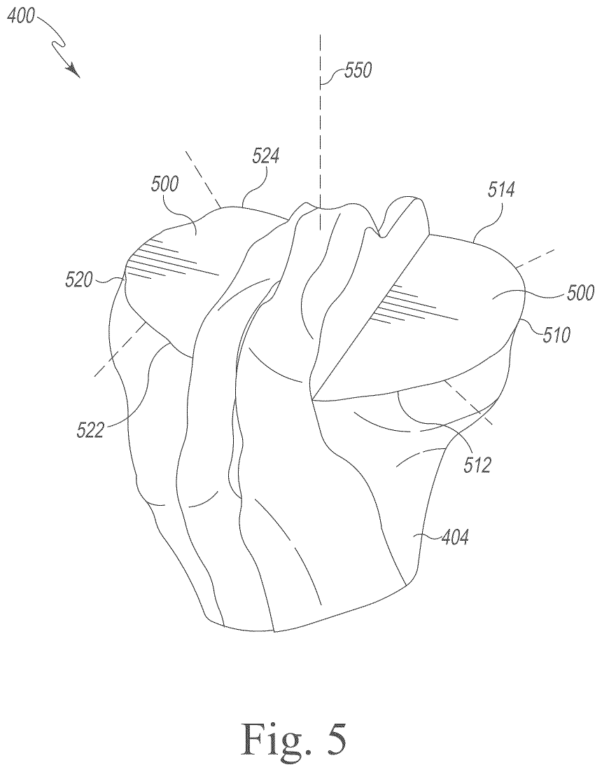

For example, as shown in FIG. 4, a virtual transverse cut 410 and a sagittal cut 412 may be performed on the medial and/or lateral side of the proximal end of a virtual tibia 404 represented by the three-dimensional statistical shape model 400. As shown in FIG. 5, the virtual resectioning of the virtual tibia 404 produce resected sections 500 on the respective resected sides of the virtual tibia 404. It should be appreciated that each of the resected sections 500 has an external edge that includes or otherwise defines an external curvature in the transverse cutting plane, which is created by the distal end of the resected virtual tibia 404. For example, as shown in FIG. 5, the resected section 500 of the medial side of the resected virtual tibia 404 has an external curvature 510 (which is located away from a central axis 550 of the virtual tibia 404), an anterior curvature 512 located anteriorly of the external curvature 510, and a posterior curvature 514 located posteriorly of the anterior curvature 516. Similarly, the resected section 500 of the lateral side of the resected virtual tibia 404 has an external curvature 520 (which is located away from the central axis 550 of the virtual tibia 404), an anterior curvature 522 located anteriorly of the external curvature 510, and a posterior curvature 514 located posteriorly of the anterior curvature 516. As discussed in more detail below, each of the curvatures 510, 512, 514, 520, 522, 524 may be used to define corresponding curvatures (i.e., corresponding curvatures of the sidewall 224) of the tibial uni-compartmental orthopaedic prosthesis 104.

In some embodiments, additional virtual resections and/or determination of other curvatures may be performed on the tibia 404 represented by the three-dimensional statistical shape model 400. For example, referring back to FIG. 4, in some embodiments, a number of additional virtual sagittal cuts 412 may be performed on the proximal end of the tibia 404. The virtual sagittal cuts 412 form corresponding “slices” of the proximal end of the tibia 404 and may be used to determine a curvature (e.g., a sagittal and/or coronal curvature) of an articular surface of the proximal end of the tibia 404. In such embodiments, again as discussed in more detail below, the determined curvature of the tibial articular surface of the virtual tibia 404 may be used to define a corresponding curvature of the tibial articular surface 204 of the tibial uni-compartmental orthopaedic prosthesis 104.

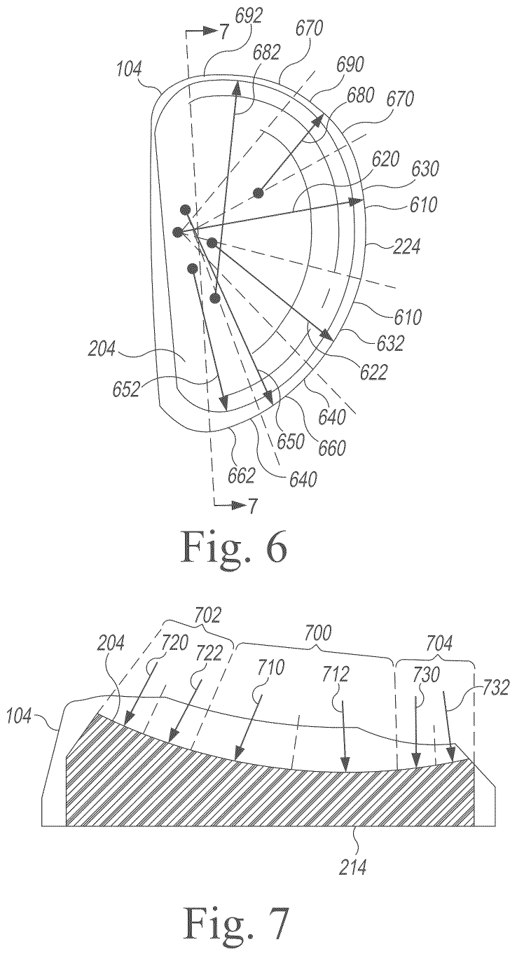

Referring now back to FIG. 3B, after the virtual bone resections and other curvature determinations have been completed in block 318, the method 300 advances to blocks 320 and 322 in which particular curvatures of the tibial uni-compartmental orthopaedic prosthesis 104 are defined based on corresponding curvatures of the three-dimensional statistical shape model 400. For example, in block 320, the curvature of the sidewall 224 of the tibial uni-compartmental orthopaedic prosthesis 104 is defined based on the determined curvatures 510, 512, 514 or 520, 522, 514 (depending on whether the tibial prosthesis 104 is a medial or lateral prosthesis) of the resected section 500 of the resected virtual tibia 404 (see FIG. 4). To do so, as shown in FIG. 6, an external curvature 610 of the sidewall 224 of the tibial uni-compartmental orthopaedic prosthesis 104 is designed to have a curvature in a transverse plane that matches or is otherwise based on the external curvature 510, 520 of the corresponding resected section 500 of the resected virtual tibia 404 (see FIG. 4). As used herein, the terms “match” and “matches” are intended to mean identical within a reference manufacturing tolerance. For example, in the illustrative embodiment, the external curvature 610 of the sidewall 224 is defined by a pair of radii 620, 622, which define corresponding external sections 630, 632 of the sidewall 224 and are tangential to each other to provide a smooth transition between the sections 630, 632 and other sections of the sidewall 224. The particular size and number of radii used to define the external curvature 610 may be based on the particular curvature 510, 520 of the resected section 500 of the virtual tibia 404 to be matched and the degree of tolerance of the “matching.” For example, in other embodiments, the external curvature 610 of the sidewall 224 of the tibial uni-compartmental orthopaedic prosthesis 104 may be defined by a single radius, by a larger number of radii, or a radius or curvature that is defined by a continuous function (e.g., a function that defines a continuously decreasing or increasing radius).

In addition to the external curvature 610, the anterior and posterior curvatures of the sidewall 224 of the tibial uni-compartmental orthopaedic prosthesis 104 may be defined in block 320. For example, as shown in FIG. 6, an anterior curvature 640 of the sidewall 224 may be defined based on the determined anterior curvature 512, 522 of the resected section 500 of the resected virtual tibia 404. To do so, the anterior curvature 640 of the sidewall 224 is designed to have a curvature in a transverse plane that matches or is otherwise based on the anterior curvature 512, 522 of the corresponding resected section 500 of the tibia 404. For example, in the illustrative embodiment, the anterior curvature 640 is defined by a pair of radii 650, 652, which define corresponding anterior sections 660, 662 of the sidewall 224 and are tangential to each other to provide a smooth transition between the sections 660, 662 and other sections of the sidewall 224. Again, the particular size and number of radii used to define the anterior curvature 640 may be based on the particular curvature 512, 522 of the resected section 500 of the virtual tibia 404 to be matched and the degree of tolerance of the “matching.” For example, in other embodiments, the anterior curvature 640 of the sidewall 224 of the tibial uni-compartmental orthopaedic prosthesis 104 may be defined by a single radius, by a larger number of radii, or a radius or curvature that is defined by a continuous function (e.g., a function that defines a continuously decreasing or increasing radius).

Similarly, a posterior curvature 670 of the sidewall 224 may be defined based on the determined posterior curvature 514, 524 of the resected section 500 of the resected virtual tibia 404. To do so, the posterior curvature 670 of the sidewall 224 is designed to have a curvature in a transverse plane that matches or is otherwise based on the posterior curvature 514, 524 of the corresponding resected section 500 of the tibia 404. For example, in the illustrative embodiment, the posterior curvature 670 is defined by a pair of radii 680, 682, which define corresponding posterior sections 690, 692 of the sidewall 224 and are tangential to each other to provide a smooth transition between the sections 690, 692 and other sections of the sidewall 224. Again, the particular size and number of radii used to define the posterior curvature 670 may be based on the particular curvature 514, 524 of the resected section 500 of the virtual tibia 404 to be matched and the degree of tolerance of the “matching.” For example, in other embodiments, the posterior curvature 670 of the sidewall 224 of the tibial uni-compartmental orthopaedic prosthesis 104 may be defined by a single radius, by a larger number of radii, or a radius or curvature that is defined by a continuous function (e.g., a function that defines a continuously decreasing or increasing radius).

Referring back to FIG. 3B, in addition to the curvature of the sidewall 224, other curvatures of the tibial uni-compartmental orthopaedic prosthesis 104 may be designed in block 316. For example, in block 322 the curvature of the tibial articular surface 204 of the tibial uni-compartmental orthopaedic prosthesis 104 is defined based on the determined curvature (in a sagittal and/or coronal plane) of tibial articular surface of the virtual tibia 404. To do so, as shown in FIG. 7, various sections of the tibial articular surface 204 of the tibial uni-compartmental orthopaedic prosthesis 104 are defined to have respective curvatures in a sagittal plane that match the determined curvature, in the sagittal plane, of the tibial articular surface of the virtual tibia 404. For example, in the illustrative embodiment, the tibial articular surface 204 includes a distal section 700 defined by a pair of radii 710, 712, which are tangential to each other to provide a smooth transition between the sections of the tibial articular surface 204. The particular size and number of radii used to define the distal section 700 may be based on the particular curvature of the tibial articular surface of the virtual tibia 404 to be matched and the degree of tolerance of the “matching.” For example, in other embodiments, the distal section 700 of the tibial articular surface 204 of the tibial uni-compartmental orthopaedic prosthesis 104 may be defined by a single radius, by a larger number of radii, or a radius or curvature that is defined by a continuous function (e.g., a function that defines a continuously decreasing or increasing radius).

The tibial articular surface 204 also includes an anterior section 702 defined by a pair of radii 720, 722, which are tangential to each other to provide a smooth transition between the sections of the tibial articular surface 204. The particular size and number of radii used to define the anterior section 702 may be based on the particular curvature of the tibial articular surface of the virtual tibia 404 to be matched and the degree of tolerance of the “matching.” For example, in other embodiments, the anterior section 702 of the tibial articular surface 204 of the tibial uni-compartmental orthopaedic prosthesis 104 may be defined by a single radius, by a larger number of radii, or a radius or curvature that is defined by a continuous function (e.g., a function that defines a continuously decreasing or increasing radius).

The illustrative tibial articular surface 204 further includes a posterior section 704 defined by a pair of radii 730, 732, which are tangential to each other to provide a smooth transition between the sections of the tibial articular surface 204. Similar to the anterior section 702, the particular size and number of radii used to define the posterior section 704 may be based on the particular curvature of the tibial articular surface of the virtual tibia 404 to be matched and the degree of tolerance of the “matching.” For example, in other embodiments, the posterior section 704 of the tibial articular surface 204 of the tibial uni-compartmental orthopaedic prosthesis 104 may be defined by a single radius, by a larger number of radii, or a radius or curvature that is defined by a continuous function (e.g., a function that defines a continuously decreasing or increasing radius).

Referring now back to FIG. 3B, after the curvatures of the tibial uni-compartmental orthopaedic prosthesis 104 have been designed in block 316, the method 300 advances to block 324 in which particular curvatures of each size of the femoral uni-compartmental orthopaedic prosthesis 102 may be defined based on corresponding curvatures of the three-dimensional statistical shape model 400. To do so, in the illustrative embodiment, virtual bone resections are performed on the virtual femur 402 represented in the three-dimensional statistical shape model 400 in block 326, which help define or indicate the shape of the curvature of interest. The virtual bone resections are representative of the actual bone cuts performed by an orthopaedic surgeon to prepare a patient's femur for receiving a femoral uni-compartmental orthopaedic prosthesis.

For example, again as shown in FIG. 4, a virtual distal cut 450 may be performed on the distal end of the virtual femur represented by the three-dimensional statistical shape model 400. The virtual resectioning of the virtual femur 402 produces resected sections 460 of the femur 402. As indicated in FIG. 4, each of the resected distal sections 460 has an anterior edge that includes or otherwise defines an anterior profile curvature in the transverse cutting plane, which is created by the distal end of the resected virtual femur 402. For example, as indicated, each resected section 460 has an internal anterior curvature 462 and an adjacent external anterior curvature 464. As discussed in more detail below, the internal and external anterior curvatures 462, 464 may be used to define corresponding curvatures of the femoral uni-compartmental orthopaedic prosthesis 102.

Although only a distal virtual resection is shown in FIG. 4, it should be appreciated that additional and/or other resection cuts may be performed in block 326. For example, a virtual anterior, a virtual posterior, and/or virtual box cuts may be performed in block 326 to define or otherwise identify corresponding curvatures of the virtual femur 402 represented in the three-dimensional statistical shape model 400, which may be subsequently used to define corresponding curvatures of the femoral uni-compartmental orthopaedic prosthesis 102 as discussed in more detail below.

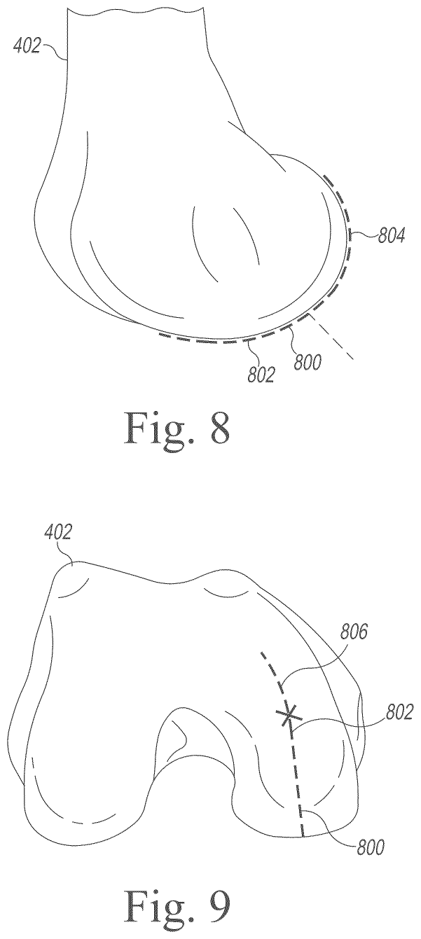

Furthermore, other curvatures of the virtual femur 402 represented in the three-dimensional statistical shape model 400 may be determined in block 326 contemporaneously with or prior to the virtual resectioning of the femur 402. For example, as shown in FIGS. 8 and 9, a femoral curve 800 of a corresponding condyle of the virtual femur 402 may be determined based on the three-dimensional statistical shape model 400. The femoral curve 800 is defined by a set of distal-most points of the corresponding condyle, with each point defining a distal most point of the condyle of the virtual femur 402 at a corresponding degree of flexion of a reference range of flexion (e.g., −10 degrees through 120 degrees of flexion). That is, at each degree of flexion (or whatever granular amount of flexion used), the corresponding condyle of the virtual femur 402 has a distal-most point, which general corresponds to the point of contact of the virtual femur 402 and the virtual tibia 404 at that particular degree of flexion. In this way, the femoral curve 800 defines a curvature of contact of the virtual femur 402 with the virtual tibia 404 through a range of flexion, transitioning to a trochlea groove for patella tracking at the anterior side of the femoral curve 800.

It should be appreciated that the femoral curve 800 may be a complex curve and may not lie on a single anatomical plane (e.g., a simple sagittal curve). For example as shown in FIGS. 8 and 9, the illustrative femoral curve includes a distal section 802, which lies generally on a sagittal plane. The femoral curve 800, however, also includes a posterior section 804 and an anterior section 806, both of which may lie on more than a single plane. As such, depending on their particular curvature, the distal section 802 and the posterior section 804 may be defined by a multi-dimensional curve or spline (e.g., a two-dimensional curve or spline).

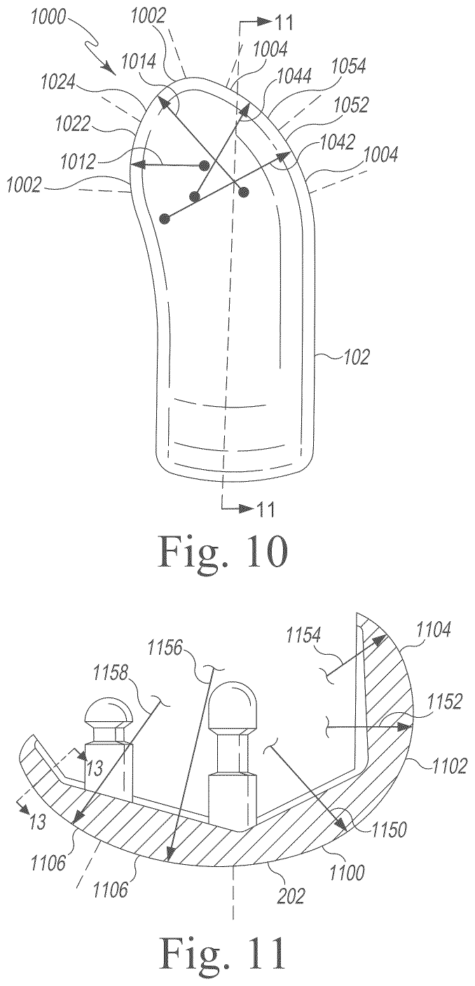

Referring again back to FIG. 3B, after the virtual bone resections and other curvature determinations have been completed in block 328, the method 300 advances to block 328 in which particular curvatures of the femoral uni-compartmental orthopaedic prosthesis 102 are defined based on corresponding curvatures of the three-dimensional statistical shape model 400. For example, as shown in FIG. 10, the curvature of an anterior sidewall 1000 of the femoral uni-compartmental orthopaedic prosthesis 102 may be defined based on the determined curvatures 462, 464 of the resected distal section 460 of the resected virtual femur 402 (see FIG. 4).

To do so, as shown in FIG. 10, an internal anterior curvature 1002 of the anterior sidewall 1000 of the femoral uni-compartmental orthopaedic prosthesis 102 is designed to have a curvature in a coronal plane that matches or is otherwise based on the internal anterior curvature 462. Similarly, an external anterior curvature 1004 of the anterior sidewall 1000 of the femoral uni-compartmental orthopaedic prosthesis 102 is designed to have a curvature in a coronal plane that matches or is otherwise based on the external anterior curvature 464. For example, in the illustrative embodiment, the internal anterior curvature 1002 is defined by a pair of radii 1012, 1014, which define corresponding internal sections 1022, 1024 of the anterior sidewall 1000. The radii 1012, 1014 are tangential to each other to provide a smooth transition between the sections 1022, 1024 and other sections of the anterior sidewall 1000. The particular size and number of radii used to define the internal anterior curvature 1002 may be based on the particular internal anterior curvature 462 of the resected distal section 460 of the virtual tibia 404 to be matched and the degree of tolerance of the “matching.” For example, in other embodiments, the internal anterior curvature 1002 of the anterior sidewall 1000 of the femoral uni-compartmental orthopaedic prosthesis 102 may be defined by a single radius or by a larger number of radii.

Similarly, in the illustrative embodiment, the external anterior curvature 1004 is defined by a pair of radii 1042, 1044, which define corresponding external sections 1052, 1054 of the anterior sidewall 1000. The radii 1042, 1044 are tangential to each other to provide a smooth transition between the sections 1052, 1054 and other sections of the anterior sidewall 1000. The particular size and number of radii used to define the external anterior curvature 1004 may be based on the particular external anterior curvature 464 of the resected distal section 460 of the virtual tibia 404 to be matched and the degree of tolerance of the “matching.” For example, in other embodiments, the external anterior curvature 1004 of the anterior sidewall 1000 of the femoral uni-compartmental orthopaedic prosthesis 102 may be defined by a single radius or by a larger number of radii.

Again, in addition to the curvature of the anterior sidewall 1000, other curvatures of the femoral uni-compartmental orthopaedic prosthesis 102 may be designed in 328. For example, as shown in FIG. 11, the curvature of the femoral articular surface 202 of the uni-condyle surface 200 of the femoral uni-compartmental orthopaedic prosthesis 102 may be defined based on the determined curvature of the femoral curve 800 of the virtual femur 402 represented in the three-dimensional statistical shape model 400. To do so, various sections of the femoral articular surface 202 are defined to have respective curvatures in one or more sagittal, coronal, and/or transverse planes that match the determined curvature, in the corresponding planes, of the determined femoral curve 800 of the virtual femur 402. For example, in the illustrative embodiment, the femoral articular surface 202 includes a distal section 1100 defined by a continually decreasing radius 1150, a mid-flexion section 1102 defined by a two-dimensional spline curve (indicated in FIG. 11 by a radius 1152), a posterior section 1104 defined by a two-dimensional radius (indicated in FIG. 11 by a radius 1154), and an anterior section 1106 defined by a pair of constant radii 1156, 1158. It should be appreciated, however, that the curvature of the articular surface 202 illustrated in FIG. 11 is only illustrative and the particular size and number of radii used to define the articular surface 202 may be based on the particular curvature of the femoral articular surface of the virtual femur 402 to be matched and the degree of tolerance of the “matching.”

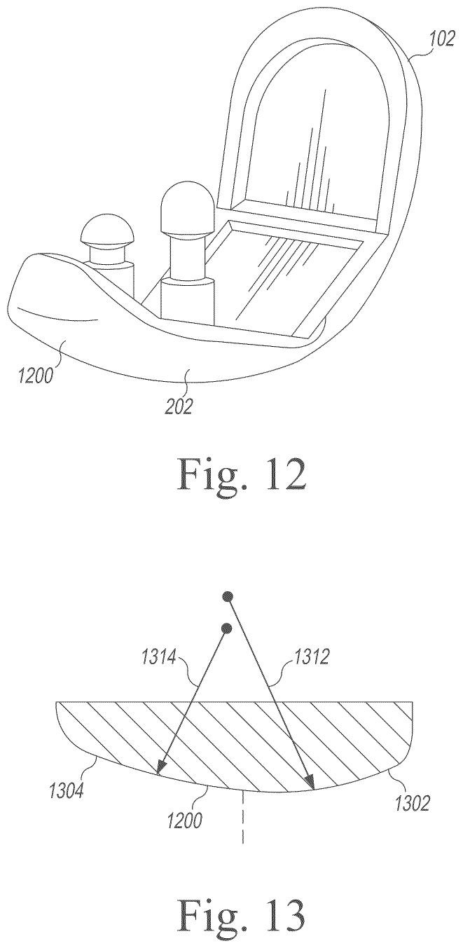

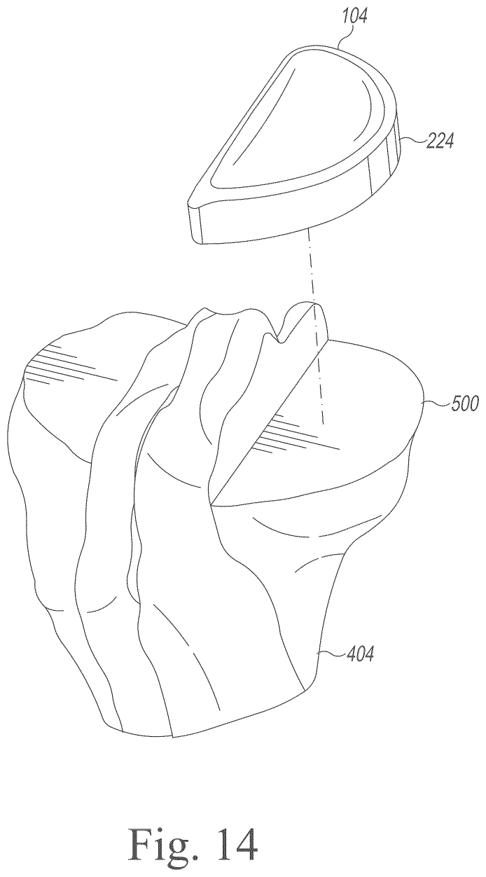

In some embodiments, the femoral articular surface 202 may include or otherwise transition to a trochlea groove section 1200 located on the anterior side of the femoral uni-compartmental orthopaedic prosthesis 102 as shown in FIG. 12. The trochlea groove section 1200 provides a surface for patella tracking of the patient's natural patella or a prosthetic patella, depending on the particular surgical procedure. Again, the trochlea groove section 1200 may be designed to have respective curvatures in one or more sagittal, coronal, and/or transverse planes that match the determined curvature, in the corresponding planes, of the determined femoral curve 800 of the virtual femur 402. For example, as shown in FIG. 13, the trochlea groove section 1200 may be designed to have a curvature in the coronal plane including a lateral section 1302 defined by a radius 1312 and a medial section 1304 defined by a radius 1314. It should be appreciated, however, that the curvature of the trochlea groove section 1200 illustrated in FIG. 13 is only illustrative and the particular size and number of radii used to define the trochlea groove section 1200 may be based on the particular curvature of the femoral articular surface of the virtual femur 402 to be matched and the degree of tolerance of the “matching.”

Referring now back to FIG. 3B, after the uni-compartmental orthopaedic prosthesis 100 has been designed based on the three-dimensional statistical shape model in block 314, the method 300 advances to block 330 of FIG. 3C. In block 330, the designed femoral and tibial uni-compartmental orthopaedic prosthesis 102, 104 may be virtually implanted into the resected virtual femur and tibia 402, 404, respectively, of the three-dimensional statistical shape model 400 to confirm the suitability and match of the designed curvatures. For example, as shown in FIG. 14, the designed tibial uni-compartmental orthopaedic prosthesis 104 may be virtually implanted into the resected virtual tibia 404 of the three-dimensional statistical shape model 400. In doing so, the designed curvature of the sidewall 224 of the tibial uni-compartmental orthopaedic prosthesis 104 may be compared to the curvature of the resected section 500 of the resected virtual tibia 404 to confirm that those curvatures match within a reference tolerance or otherwise meet defined or reference criteria such as area coverage, degree of over-hang, degree of under-hang, and/or other measurements of fit (e.g., based on patient bone scans). In this way, the femoral and tibial uni-compartmental orthopaedic prosthesis 102, 104 can be designed for an average patient's femur and tibia across a range of sizes.

Referring back to FIG. 3C, after the femoral and/or tibial uni-compartmental orthopaedic prosthesis 102, 104 has been virtually implanted into the respective virtual femur 402 or tibia 404, it is determined whether the design of the prosthesis 102, 104, is acceptable in block 332 (e.g., do the designed curvatures match the corresponding curvatures of the three-dimensional statistical shape model 400 within a reference tolerance). If not, the method 300 loops back to bock 314 of FIG. 3C in which the femoral and/or tibial uni-compartmental orthopaedic prosthesis 102, 104 is redesigned to have curvatures that better match the corresponding curvatures of the three-dimensional statistical shape model 400. If, however, the design of the three-dimensional statistical shape model 400 is acceptable in block 332, the method 300 advances to block 334. In block 334, the designed femoral uni-compartmental orthopaedic prosthesis 102 and/or the designed tibial uni-compartmental orthopaedic prosthesis 104 are manufactured. Any suitable manufacturing technique or methodology may be used to manufacture the femoral and/or tibial uni-compartmental orthopaedic prosthesis 102, 104 to include the curvatures designed based on the three-dimensional statistical shape model 400 as discussed above.

While the disclosure has been illustrated and described in detail in the drawings and foregoing description, such an illustration and description is to be considered as illustrative and not restrictive in character, it being understood that only illustrative embodiments have been shown and described and that all changes and modifications that come within the spirit of the disclosure are desired to be protected.

There are a plurality of advantages of the present disclosure arising from the various features of the methods, apparatuses, and/or systems described herein. It will be noted that alternative embodiments of the methods, apparatuses, and systems of the present disclosure may not include all of the features described yet still benefit from at least some of the advantages of such features. Those of ordinary skill in the art may readily devise their own implementations of the methods, apparatuses, and systems that incorporate one or more of the features of the present invention and fall within the spirit and scope of the present disclosure as defined by the appended claims.

1. A method for designing a uni-compartmental orthopaedic prosthesis comprising:
generating a three-dimensional statistical shape model of a knee joint, wherein the three-dimensional statistical shape model is indicative of an average knee joint of a pool of patient participants;
determining a curvature of a section of a bone of the knee joint represented by the three-dimensional statistical shape model; and
designing the uni-compartmental orthopaedic prosthesis for the bone of the knee joint based on the three-dimensional statistical shape model, wherein the uni-compartmental orthopaedic prosthesis includes a section corresponding to the section of the bone of the knee joint represented by the three-dimensional statistical shape model and wherein designing the uni-compartmental orthopaedic prosthesis comprises defining a curvature of the section of the uni-compartmental orthopaedic prosthesis to match the curvature of the corresponding section of the bone of the knee joint represented by the three-dimensional statistical shape model.

2. The method of claim 1, wherein determining the curvature of the section of the bone of the knee joint comprises performing a virtual bone resection on the bone of the knee joint represented by the three-dimensional statistical shape model to determine the curvature.

3. The method of claim 2, wherein the uni-compartmental orthopaedic prosthesis comprises a tibial uni-compartmental orthopaedic prosthesis, and
wherein performing the virtual bone resection comprises performing a virtual bone resection in a transverse plane of a proximal end of a tibia of the knee joint represented by the three-dimensional statistical shape model to form a resected section of the tibia in the transverse plane, wherein the curvature of the section comprises a lateral external curvature or a medial external curvature of the resected section of the proximal end of the tibia in the transverse plane.

4. The method of claim 3, wherein defining the curvature of the section of the uni-compartmental orthopaedic prosthesis comprises matching a curvature of an external sidewall of the tibial uni-compartmental orthopaedic prosthesis in the transverse plane to the lateral external curvature or the medial external curvature of the resected section of the tibia in the transverse plane.

5. The method of claim 3, wherein the curvature of the section further comprises an anterior curvature and a posterior curvature of the resected section of the tibia in the transverse plane.

6. The method of claim 5, wherein defining the curvature of the section of the uni-compartmental orthopaedic prosthesis comprises matching (i) a curvature of an anterior sidewall of the tibial uni-compartmental orthopaedic prosthesis in the transverse plane to the anterior curvature of the resected section of the tibia in the transverse plane and (ii) a curvature of a posterior sidewall of the tibial uni-compartmental orthopaedic prosthesis in the transverse plane to the posterior curvature of the resected section of the tibia in the transverse plane.

7. The method of claim 2, wherein the uni-compartmental orthopaedic prosthesis comprises a tibial uni-compartmental orthopaedic prosthesis, and
wherein determining the curvature of the section of the bone of the knee joint comprises determining a curvature of an articular surface of a tibia of the knee joint represented by the three-dimensional statistical shape model in a sagittal plane.

8. The method of claim 7, wherein defining the curvature of the section of the uni-compartmental orthopaedic prosthesis comprises matching a curvature of an articular surface of the tibial uni-compartmental orthopaedic prosthesis in the sagittal plane to the curvature of the articular surface of the tibia of the knee joint represented by the three-dimensional statistical shape model in the sagittal plane.

9. The method of claim 2, wherein the uni-compartmental orthopaedic prosthesis comprises a femoral uni-compartmental orthopaedic prosthesis, and
wherein determining the curvature of the section of the bone of the knee joint comprises performing a virtual bone resection in a transverse plane of a distal end of a femur of the knee joint represented by the three-dimensional statistical shape model to form a resected section of the femur in the transverse plane, wherein the curvature of the section comprises an external anterior curvature and an internal anterior curvature of the resected section of the distal end of the femur in the transverse plane.

10. The method of claim 9, wherein defining the curvature of the section of the uni-compartmental orthopaedic prosthesis comprises matching (i) a curvature of an external anterior sidewall of the femoral uni-compartmental orthopaedic prosthesis in the transverse plane to the external anterior curvature of the resected section of the femur in the transverse plane and (ii) a curvature of an internal anterior sidewall of the femoral uni-compartmental orthopaedic prosthesis in the transverse plane to the internal anterior curvature of the resected section of the femur in the transverse plane.

11. The method of claim 1, wherein the uni-compartmental orthopaedic prosthesis comprises a femoral uni-compartmental orthopaedic prosthesis, and
wherein determining the curvature of the section of the bone of the knee joint represented by the three-dimensional statistical shape model comprises defining a femoral curve formed from a set of distal-most points of a condyle of a femur of the knee joint represented by the three-dimensional statistical shape model, wherein each of the distal-most points defines a distal-most point of the condyle of the femur at a corresponding degree of flexion.

12. The method of claim 11, wherein the femoral curve includes a distal section corresponding to a distal section of the condyle of the femur of the knee joint represented by the three-dimensional statistical shape model that is defined by a continually decreasing radius of curvature.

13. The method of claim 12, wherein defining the curvature of the section of the uni-compartmental orthopaedic prosthesis comprises matching a distal curvature, when viewed in a sagittal plane through the range of flexion, of a femoral articular surface of the femoral uni-compartmental orthopaedic prosthesis to the distal section of the femoral curve of the femur of the knee joint represented by the three-dimensional statistical shape model.

14. The method of claim 12, wherein the femoral curve includes (i) a posterior section corresponding to a posterior section of the condyle of the femur of the knee joint represented by the three-dimensional statistical shape model that is defined by a two-dimensional radius and (ii) a mid-flexion section, located between the distal section and the posterior section, corresponding to a mid-flexion section of the condyle of the femur of the knee joint represented by the three-dimensional statistical shape model that is defined by a two-dimensional spline curve.

15. The method of claim 14, wherein defining the curvature of the section of the uni-compartmental orthopaedic prosthesis comprises matching (i) a curvature of a posterior section of a femoral articular surface of the femoral uni-compartmental orthopaedic prosthesis to the posterior section of the femoral curve of the femur of the knee joint represented by the three-dimensional statistical shape model and (ii) a curvature of a mid-flexion section of the femoral articular surface of the femoral uni-compartmental orthopaedic prosthesis, located between the distal section and the posterior section, to the mid-flexion section of the femoral curve of the femur of the knee joint represented by the three-dimensional statistical shape model.

16. A tibial uni-compartmental orthopaedic prosthesis comprising:
an anterior end and a posterior end opposite the anterior end;
an articular surface extending from the anterior end of the posterior end and configured to articulate with a corresponding condyle of a natural or prosthetic femur;
a bottom surface, opposite the articular surface, extending form the anterior end to the posterior end; and
an external sidewall extending from the bottom surface to the articular surface and from the anterior end to the posterior end, wherein the external sidewall has a curvature, when viewed in a transverse plane, that matches a lateral external curvature or a medial external curvature of a resected section, in the transverse plane, of a proximal end of a tibia represented in a three-dimensional statistical shape model of a knee joint, wherein the three-dimensional statistical shape model is indicative of an average knee joint of a pool of patient participants.

17. The tibial uni-compartmental orthopaedic prosthesis of claim 16, wherein the anterior end includes an anterior end sidewall extending from the bottom surface to the articular surface that has a curvature, when viewed in the transverse plane, that matches an anterior curvature of the resected section, in the transverse plane, of the proximal end of the tibia represented in the three-dimensional statistical shape model.

18. The tibial uni-compartmental orthopaedic prosthesis of claim 16, wherein the posterior end includes a posterior end sidewall extending from the bottom surface to the articular surface that has a curvature, when viewed in the transverse plane, that matches a posterior curvature of the resected section, in the transverse plane, of the proximal end of the tibia represented in the three-dimensional statistical shape model.

19. A femoral uni-compartmental orthopaedic prosthesis comprising:
a bottom surface configured to be coupled to a surgically-prepared distal end of a patient' femur; and
a uni-condyle surface opposite the bottom surface and having a femoral articular surface configured to articulate with a corresponding articular surface a natural or prosthetic tibia,
wherein the femoral articular surface includes a femoral curve defined by a first set of distal-most points, each of which defines a distal-most point of the femoral articular surface at a corresponding degree of flexion, and wherein the femoral curve matches a virtual femoral curve of a condyle of a femur represented in a three-dimensional statistical shape model of a knee joint, wherein the virtual femoral curve is defined by a second set of distal-most points, each of which defines a distal-most point of a femoral condyle of the femur at a corresponding degree of flexion, and wherein the three-dimensional statistical shape model is indicative of an average knee joint of a pool of patient participants.

20. The femoral uni-compartmental orthopaedic prosthesis of claim 19, further comprising an anterior end having an external sidewall and an internal sidewall, each sidewall extending from the bottom surface to the uni-condyle surface,
wherein the external sidewall has a curvature, when viewed in a transverse plane, that matches an external anterior curvature of a resected section, in the transverse plane, of a distal end of the femur represented in the three-dimensional statistical shape model of the knee joint, and
wherein the internal sidewall has a curvature, when viewed in a transverse plane, that matches an internal anterior curvature of a resected section, in the transverse plane, of the distal end of the femur represented in a three-dimensional statistical shape model of a knee joint.
